(19) United States
(12) Patent Application Publication
(10) Pub. No.: US 2017/0049749 A1
(43) Pub. Date: Feb. 23, 2017

(54) METHODS FOR TREATING BURNS USING ALLANTOIN

(71) Applicant: Scioderm.Inc., Durham, NC (US)

(72) Inventor: Robert RYAN, Durham, NC (US)

(73) Assignee: Scioderm.Inc., Durham, NC (US)

(21) Appl. No.: 15/118,782

(22) PCT Filed: Feb. 13, 2015
(86) PCT No.: PCT/US15/15781

§ 371 Date: Aug. 12, 2016

Related U.S. Application Data
(60) Provisional application No. 61/940,069, filed on Feb. 14, 2014.

(51) Int. Cl. A61K 31/4166; A61K 9/00

(52) U.S. Cl. CPC A61K 31/4166; A61K 9/0014

(57) ABSTRACT

Embodiments of the present invention relate generally to compositions comprising an effective amount of allantoin that can be used to treat skin burns. The compositions are preferably formulated as topical formulations, e.g., oil-in-water emulsions. Embodiments herein provide formulations and methods for treating skin burns using allantoin in an amount from about 2.5% to about 15.0% by weight.

### CROSS-REFERENCE TO RELATED APPLICATIONS

This application claims priority benefit to U.S. Provisional Appl. No. 61/940,069, filed on Feb. 14, 2014, the contents of which are hereby incorporated by reference in their entirety.

### BACKGROUND OF THE INVENTION

Globally, about 11 million people seek medical treatment for burns, and according to the World Health Organization, 300,000 die from burns annually throughout the world (not including cases from war). In the United States, approximately 4% of those admitted to a burn center die from their injuries. The long-term outcome is primarily related to the size of burn and the age of the person affected. Burns requiring comfort treatment are estimated to involve about 10% of the population. Therefore, there is a need for compositions for treating burns.

A burn is a type of injury caused by, e.g., heat, electricity, chemicals, friction, or radiation. Most burns affect only the skin, namely: the epidermis and the dermis. Burns that affect only the superficial skin are generally known as superficial or first-degree burns. When damage penetrates into some of the underlying layers, it is generally referred to as a partial-thickness or second-degree burn. In a full-thickness or third-degree burn, the injury extends to all layers of the skin. A fourth-degree burn additionally involves injury to deeper tissues, such as muscle or bone. The treatment required depends on the severity of the burn. Several factors are taken into account for evaluating the seriousness of a burn, e.g., the surface area affected, the depth, the localization and also the cause of the burn. Most burns can be managed on an outpatient basis by a primary care physician. See, e.g., Lloyd et al., American Family Physician 85(1):25-32 (2012).

Compositions intended for treating burns are known. For example, superficial burns can be treated with topical application of lotions, honey, aloe vera, or antibiotic ointment. Partial-thickness burns can be treated with a topical antimicrobial agent or an absorptive occlusive dressing to help reduce pain, promote healing, and prevent wound desiccation. Topical silver sulfadiazine is a standard treatment; however, newer occlusive dressings can provide faster healing and are often more cost-effective. Id.

Nevertheless, it is generally observed that the time for total healing of a first-degree or second-degree skin (also referred to as cutaneous) burn can be up to two weeks.

Thus, there is a need for alternative pharmaceutical compositions and methods for treating skin burns.

### BRIEF SUMMARY OF THE INVENTION

Embodiments of the present invention relate generally to compositions comprising an effective amount of allantoin that can be used to treat skin burns. The compositions are preferably formulated as topical formulations, e.g., oil-in-water emulsions.

In one aspect, embodiments describe methods of treating skin burns comprising administering a composition comprising from about 2.5% to about 15% allantoin and a pharmaceutically acceptable excipient.

In some embodiments, the skin burn is caused by heat, electricity, chemicals, friction, or radiation. In some embodiments the skin burn is caused by heat (also referred to as a thermal burn).

In some embodiments, the skin burn is a first degree burn or a second degree burn.

In another aspect, embodiments describe methods of treating burns comprising administering a composition comprising about 2.5% to about 15%, about 3% to about 15%, about 3% to about 10%, about 3% to about 9%, about 6% to about 15%, about 6% to about 10% or about 6% to about 9% allantoin and a pharmaceutically acceptable excipient. In another aspect, embodiments describe methods of treating burns comprising administering a composition comprising about 3.0% allantoin and a pharmaceutical excipient. In another aspect, embodiments describe methods of treating burns comprising administering a composition comprising about 6.0% allantoin and a pharmaceutical excipient. In another aspect, embodiments describe methods of treating burns comprising administering a composition comprising about 9.0% allantoin and a pharmaceutical excipient.

In some embodiments, the composition is formulated for topical administration.

In some embodiments, the amount of allantoin is not 2.0% by weight or less of the composition. In embodiments, the composition further comprises an emollient, an emulsifier, a solvent, a pH modifier, a solubilizing agent, an antioxidant, a preservative, a chelating agent, an additive, a viscosity agent or a combination thereof. In some embodiments, the formulation comprises allantoin, water, cetyl alcohol, stearyl alcohol, beeswax, sodium lauryl sulfate in an about 30% solution, citric acid, lanolin oil, propylene glycol, cod liver oil (and/or mineral oil), butylated hydroxytoluene, methylparaben, propylparaben or a combination thereof. In some embodiments, the formulation comprises allantoin in an amount from about 2.5% to about 15%, water in an amount from about 40% to about 90%; cetyl alcohol in an amount from about 0.5% to about 15%; stearyl alcohol in an amount from about 1% to about 3%; beeswax in an amount from about 1.5% to about 3%; sodium lauryl sulfate in a 30% solution in an amount from about 1.5% to about 3%; citric acid in an amount from about 0.5% to about 0.2%; lanolin oil in an amount from about 5% to about 15%; propylene glycol in an amount from about 2% to about 8%; cod liver oil (and/or mineral oil) in an amount from about 0.05% to about 5%; butylated hydroxytoluene in an amount from about 0.05% to about 1%; methylparaben in an amount from about 0.05% to about 0.5%; propylparaben in an amount from about 0.05% to about 0.5% by weight of the formulation or a combination thereof. In embodiments, the formulation optionally includes tetrasodium EDTA. In embodiments, the tetrasodium EDTA may be present in an amount from about 0.05% to about 0.5% by weight of the formulation. In embodiments, the formulation optionally includes a fragrance. In some embodiments, allantoin may be present in an amount from about 1.5% to about 15%, from about 2.0% to about 15%, from about 2.5% to about 15% or from about 3.0% to about 15% by weight of the formulation.

In one aspect, formulations of allantoin for treating burns comprise from about 2.5% to about 15% of allantoin by weight and a pharmaceutically acceptable excipient are provided. In some embodiments, the amount of allantoin is not 1.5% by weight or less of the composition. In some embodiments, the amount of allantoin is not 2.0% by weight or less of the composition. In embodiments, the formulation further comprises an emollient, an emulsifier, a solvent, a pH modifier, a solubilizing agent, an antioxidant, a preservative, a chelating agent, an additive, a viscosity agent or a combination thereof. In some embodiments, the formulation comprises, allantoin in an amount from about 2.5% to about 15% by weight of the formulation, water, cetyl alcohol, stearyl alcohol, beeswax, sodium lauryl sulfate in an about 30% solution, citric acid, lanolin oil, propylene glycol, cod liver oil (and/or mineral oil), butylated hydroxytoluene, methylparaben, propylparaben or a combination thereof. In an embodiment, the formulation may comprise allantoin in an amount of from about 2.5% to about 15%; water in an amount from about 40% to about 90%; cetyl alcohol in an amount from about 0.5% to about 15%; stearyl alcohol in an amount from about 1% to about 3%; beeswax in an amount from about 1.5% to about 3%; sodium lauryl sulfate in a 30% solution in an amount from about 1.5% to about 3%; citric acid in an amount from about 0.5% to about 0.2%; lanolin oil in an amount from about 5% to about 15%; propylene glycol in an amount from about 2% to about 8%; tetrasodium EDTA in an amount from about 0.05% to about 0.5%; cod liver oil (and/or mineral oil) in an amount from about 0.05% to about 5%; butylated hydroxytoluene in an amount from about 0.05% to about 1%; methylparaben in an amount from about 0.05% to about 0.5%; propylparaben in an amount from about 0.05% to about 0.5% by weight of the formulation or a combination thereof. In embodiments, the formulation optionally includes tetrasodium EDTA. In embodiments, the tetrasodium EDTA may be present in an amount from about 0.05% to about 0.5% by weight of the formulation. In embodiments, the formulation optionally includes a fragrance. In some embodiments, allantoin may be present in an amount from about 1.5% to about 15%, from about 2.0% to about 15%, from about 2.5% to about 15% or from about 3.0% to about 15% by weight of the formulation. In some embodiments, the amount of allantoin is not 1.5% by weight or less of the composition. In some embodiments, the amount of allantoin is not 2.0% by weight or less of the composition.

In another aspect, formulations of allantoin for treating burns comprise about 3.0% of allantoin, water, cetyl alcohol, stearyl alcohol, beeswax, sodium lauryl sulfate in an about 30% solution, citric acid, lanolin oil, propylene glycol, tetrasodium EDTA, cod liver oil (and/or mineral oil), butylated hydroxytoluene, methylparaben, and propylparaben are provided. In another aspect, formulations of allantoin comprising about 6.0% of allantoin, water, cetyl alcohol, stearyl alcohol, beeswax, sodium lauryl sulfate in an about 30% solution, citric acid, lanolin oil, propylene glycol, tetrasodium EDTA, cod liver oil (and/or mineral oil), butylated hydroxytoluene, methylparaben, and propylparaben are provided. In another aspect, formulations of allantoin comprising about 9.0% of allantoin, water, cetyl alcohol, stearyl alcohol, beeswax, sodium lauryl sulfate in a about 30% solution, citric acid, lanolin oil, propylene glycol, tetrasodium EDTA, cod liver oil (and/or mineral oil), butylated hydroxytoluene, methylparaben, and propylparaben are provided.

These and other features provided by the present disclosure are set forth herein.

### DESCRIPTION OF DRAWINGS

For a fuller understanding of the nature and advantages of the present invention, reference should be had to the following detailed description taken in connection with the accompanying drawings, in which:

FIG. 1 illustrates exemplary formulations of allantoin according to embodiments disclosed herein.

### DETAILED DESCRIPTION

Before the present compositions and methods are described, it is to be understood that this application is not limited to the particular processes, compositions, or methodologies described, as these may vary. Unless defined otherwise, all technical and scientific terms used herein have the same meanings as commonly understood by one of ordinary skill in the art. Although any methods and materials similar or equivalent to those described herein can be used in the practice or testing of embodiments of the present application, the preferred methods, devices, and materials are now described.

As used herein and in the appended claims, the singular forms “a”, “an”, and “the” include plural reference unless the context clearly dictates otherwise. Thus, for example, reference to “an additive” is a reference to one or more additives and equivalents thereof known to those skilled in the art, and so forth.

As used herein, the term “about” means plus or minus 10% of the numerical value of the number with which it is being used. In other embodiments, the term “about” means plus or minus 1% of the numerical value of the number with which it is being used. For example, about 50% means in the range of 45%-55% or 49.5%-50.5% as described herein.

The term “inhibiting” includes the administration of a compound of the present application to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, “room temperature” means an indoor temperature of from about 20° C. to about 25° C. (68 to 77° F.).

Unless otherwise indicated, the term “skin” means that outer integument or covering of the body, consisting of the dermis and the epidermis and resting upon subcutaneous tissue.

The term “improves” is used to convey that a composition of the application changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, decreased spread of blisters, decreased redness, reduction of scarring, healing of blisters, a reduction in symptoms including, but not limited to, pain, inflammation, itching, or other symptoms associated with skin burns.

As used herein, the term “sole active ingredient” means that the active ingredient or active compound (identified as such) is the only effective therapeutic in the formulation to treat the condition (e.g., skin burn). In some embodiments, allantoin is the sole active ingredient in formulation for the treatment of a skin burn.

As used herein, the term “therapeutic” means an agent utilized to treat, combat, ameliorate, prevent or improve an unwanted condition of a patient. In part, embodiments of the present application are directed to the treatment of skin burns.

A “therapeutically effective amount” or “effective amount” of a composition is a predetermined amount calculated to achieve the desired effect, i.e., to enhance appearance of skin, to alleviate inflammation or blisters, or to prevent the skin condition from worsening. The activity contemplated by the present methods includes both medical therapeutic and/or prophylactic treatment, as appropriate. The specific dose of a compound administered according to this application to obtain therapeutic and/or prophylactic effects will, of course, be determined by the particular circumstances surrounding the case, including, for example, the compound administered, the route of administration, and the condition being treated. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the application in any way. A therapeutically effective amount of compound of this application is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, or to obtain beneficial or desired clinical results related to treatment of the skin burns. For the purposes of this application, beneficial or desired clinical results include, but are not limited to, alleviation of symptoms (e.g., symptoms associated with skin burns); diminishment of the extent of the condition; stabilization (i.e., not worsening) of the state of the condition; amelioration of the condition; and remission (whether partial or total), whether detectable or undetectable, or enhancement or improvement of the condition. Treatment includes eliciting a clinically significant response without excessive levels of side effects.

For example, in some aspects, the application is directed to a method of treating skin burns using a pharmaceutical composition comprising allantoin, as disclosed herein, and a pharmaceutically acceptable carrier or diluent, or an effective amount of a pharmaceutical composition comprising allantoin, as disclosed herein.

### I. Burns

A burn is a type of injury to skin (or flesh) due to, e.g., heat, electricity, chemicals, friction, or radiation. In particular, burns can arise from various causes, such as, e.g., contact with a solid, liquid or gaseous heat source (thermal burn); radiation from a heat source (thermal burn); certain medical treatments such as radiotherapy; contact with cold (frostbite); an electrocution (electrical burn); contact with a chemical product (chemical burn); and friction (friction burn which is generally likened to a thermal burn). In certain embodiments, the methods of this application are directed to treating the skin of a subject suffering from a burn due to one or more of the causes disclosed herein.

Burns that affect only the superficial skin are known as superficial or first-degree burns. When damage penetrates into some of the underlying layers, it is a partial-thickness or second-degree burn. In a full-thickness or third-degree burn, the injury extends to all layers of the skin. A fourth-degree burn additionally involves injury to deeper tissues, such as muscle or bone. In certain embodiments, the methods of this application are directed to treating the skin of a subject suffering from a first-degree burn, a second-degree burn, a third-degree burn, a fourth degree burn, or any combination thereof.

Skin (also referred to as cutaneous) burns are usually listed according to their degrees of seriousness. A first-degree burn is generally limited to an erythema, and affects only the surface layers of the epidermis. A second-degree burn “A” affects the epidermis and also a part of the dermis. These burns manifest themselves through the appearance of erythema and also of phlyctenae at the surface thereof. This type of burn may be responsible for more or less acute pain depending on the degree to which the nerves are affected. The rupturing of blood capillaries may already be associated therewith. A deep second-degree burn “B” can extend beyond the dermis and reach the subcutaneous soft tissues. A third-degree burn causes total destruction or vitrification of the epidermis and of the dermis. This type of burn often damages the subcutaneous tissues such as the vascular tissue, the muscles and the nerves. In order to treat this type of burn, it is customary practice to perform skin grafts since no epidermal cicatrization is theoretically possible. In some embodiments the application is directed to treating a skin graft of a subject treated for a skin burn, e.g., a third-degree burn. The fourth-degree burn affects the muscles and can even extend to the bones. In this case, the appearance of the skin is said to be “carbonized” and the recommended treatment may be amputation. In this case, the patient's vital prognosis is then often involved depending on the age of the patient and the surface area affected.

At temperatures greater than 44° C. (111° F.), proteins begin losing their three-dimensional shape and start breaking down. This results in cell and tissue damage. Many of the direct health effects of a burn are secondary to disruption in the normal functioning of the skin. They include disruption of the skin's sensation, ability to prevent water loss through evaporation, and ability to control body temperature. Disruption of cell membranes causes cells to lose potassium to the spaces outside the cell and to take up water and sodium.

In large burns (over 30% of the total body surface area), there is a significant inflammatory response. This results in increased leakage of fluid from the capillaries, and subsequent tissue edema. This causes overall blood volume loss, with the remaining blood suffering significant plasma loss, making the blood more concentrated. Poor blood flow to organs such as the kidneys and gastrointestinal tract may result in renal failure and stomach ulcers. Increased levels of catecholamines and cortisol may result in a hypermetabolic state which can last for years. This is associated with increased cardiac output, metabolism, a fast heart rate, and poor immune function.

Burns can be classified by depth, mechanism of injury, extent, and associated injuries. The most commonly used classification is based on the depth of injury. The depth of a burn is usually determined via examination, although a biopsy may also be used.

The size of a burn is measured as a percentage of total body surface area (TBSA) affected by partial thickness or full thickness burns. First-degree burns that are only red in color and are not blistering are not included in this estimation. Most burns (70%) involve less than 10% of the TBSA.

There are a number of methods to determine the TBSA, including the “rule of nines”, Lund and Browder charts, and estimations based on a person's palm size. The rule of nines is easy to remember but only accurate in people over 16 years of age. More accurate estimates can be made using Lund and Browder charts, which take into account the different proportions of body parts in adults and children. The size of a person's handprint (including the palm and fingers) is approximately 1% of their TBSA.

TABLE 1
American Burn Association Severity Classification

| Minor Burn | Moderate Burn | Major Burn |
|---|---|---|
| Adult < 10% TBSA | Adult 10-20% TBSA | Adult > 20% TBSA |
| Young or old < | Young or old | Young or old > |
| 5% TBSA | 5-10% TBSA | 10% TBSA |
| <2% full thickness | 2-5% full thickness | >5% full thickness |
| burn | burn | burn |
| | High voltage injury | High voltage burn |
| | Possible inhalation injury | Known inhalation injury |
| | Circumferential burn | Significant burn to face, |
| | | joints, hands or feet |
| | Other health problems | Associated injuries |

In order to determine the need for referral to a specialized burn unit, the American Burn Association devised a classification system (Table 1). Under this system, burns can be classified as major, moderate and minor. This is assessed based on a number of factors, including total body surface area affected, the involvement of specific anatomical zones, the age of the person, and associated injuries. Minor burns can typically be managed at home, moderate burns are often managed in hospital, and major burns are managed by a burn center.

In certain embodiments, the methods of this application are directed to treating the skin of a subject suffering from a minor burn, a moderate burn, a major burn, or any combination thereof.

### II. Compounds

The structure of allantoin is:

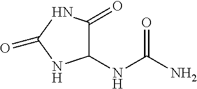 [SMILES: NC(=O)NC1NC(=O)NC1=O]

Encompassed within this disclosure is all forms of allantoin, or a salt thereof, including, but not limited to, crystals, polymorphs, clathrates, solvates, hydrates, amorphous forms, co-crystals, and anhydrous forms. As used herein, “allantoin” includes salts thereof (as described below), crystals, polymorphs, clathrates, solvates, hydrates, amorphous forms, co-crystals, and anhydrous forms unless otherwise specified.

Embodiments of the present disclosure also relate to the salts of allantoin. The acids which are used to prepare the salts of the aforementioned compound are those which form non-toxic salts, i.e., salts containing pharmacologically acceptable anions, such as the hydrochloride, acetate, trifluoroacetic acid, tosylate, picrate, hydrobromide, hydroiodide, nitrate, sulfate, bisulfate, phosphate, acid phosphate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate salts.

Many allantoin compositions are prepared as emulsions, particularly oil-in-water emulsions. One emulsifier system used with such compositions is a combination of sodium lauryl sulfate and beeswax. Although solutions of sodium lauryl sulfate are alkaline with an approximate pH of 9.5, the simultaneous use of beeswax with its organic acids produces a complex neutralized system with a pH of about 6.8 to about 7.5. However, in such a system with a pH range of 6.8 to 7.5, allantoin degrades significantly with time and in accelerated stability tests at 40° C. Because preparations designed for application to the skin are typically stored by users at room temperature, and room temperatures can fluctuate with climactic conditions, such a degree of stability is undesirable. Therefore, there is a need for an oil-in-water emulsified composition containing allantoin in which the stability of allantoin is increased.

In general, embodiments herein describe a method of treating skin burns comprising applying to the skin an allantoin comprising composition in a therapeutically effective amount. It was unexpectedly found that stabilized oil-in-water emulsions containing allantoin optionally plus other pharmaceutically acceptable ingredients as described herein provide a high degree of healing for skin burns. Administration of formulations of allantoin described in embodiments herein may cause a reduction in symptoms associated with skin burns such as, without limitation, pain, scarring, inflammation, redness, itching, blisters, or other symptoms associated with skin burns. The allantoin-containing composition comprises an oil-in-water emulsion as may be described below.

Furthermore, formulations of allantoin in embodiments described herein may impart long lasting stability at room temperature (where refrigeration is not needed) to the formulation. In some embodiments, the formulation may be stable for about 4 to about 10 years, for about 4 to about 8 years, for about 4 to about 7 years, for about 4 to about 6 years, for about 5 to about 10, for about 5 to about 8 years, for about 5 to about 7 years, for about 5 to about 6 years, for about 6 to about 10 years, for about 6 to about 8 years, or for about 6 to about 7 years. In some embodiments, stability may include, without limitation, physical stability, chemical stability, resistance to microbial agents or combinations thereof. In some embodiments, stability refers to a stability of allantoin. In some embodiments, stability refers to a period where there is no degradation of allantoin at room temperature. In some embodiments, stability refers to a period where there may be about 1% or less degradation of allantoin at room temperature. In some embodiments, stability refers to a period where there is no decrease in concentration. In some embodiments, stability refers to a period where there is less than about 1% decrease in concentration. In some embodiments, stability refers to a period of resistance to microbiological growth at room temperature. In some embodiments, stability refers to a period where the formulation falls within the normal bioburden ranges for said formulation at room temperature. In some embodiments, the formulations of allantoin in embodiments described herein may impart better absorption of the active pharmaceutical across a skin barrier. In some embodiments, the skin barrier comprises intact skin. In some embodiments, the formulations of allantoin in embodiments described herein may deliver more allantoin across intact skin barrier than formulations of prior art.

Embodiments of the present disclosure relate to formulations of allantoin and methods of treating skin burns. In some embodiments, the formulation comprises about 0.5% or more of allantoin and a pharmaceutically acceptable excipient. In some embodiments, the formulation consists essentially of about 0.5% or more of allantoin and a pharmaceutically acceptable excipient. In some embodiments, the formulation consists of about 0.5% or more of allantoin and a pharmaceutically acceptable excipient. In some embodiments, the formulation comprises about 1.5% or more of allantoin and a pharmaceutically acceptable excipient. In some embodiments, the formulation consists essentially of about 1.5% or more of allantoin and a pharmaceutically acceptable excipient. In some embodiments, the formulation consists of about 1.5% or more of allantoin and a pharmaceutically acceptable excipient. In some embodiments, the formulation comprises about 2.0% or more of allantoin and a pharmaceutically acceptable excipient. In some embodiments, the formulation consists essentially of about 2.0% or more of allantoin and a pharmaceutically acceptable excipient. In some embodiments, the formulation consists of about 2.0% or more of allantoin and a pharmaceutically acceptable excipient. In some embodiments, the formulation comprises about 2.5% or more of allantoin and a pharmaceutically acceptable excipient. In some embodiments, the formulation consists essentially of about 2.5% or more of allantoin and a pharmaceutically acceptable excipient. In some embodiments, the formulation consists of about 2.5% or more of allantoin and a pharmaceutically acceptable excipient. In some embodiments, the formulation comprises about 3.0% or more of allantoin and a pharmaceutically acceptable excipient. In some embodiments, the formulation consists essentially of about 3.0% or more of allantoin and a pharmaceutically acceptable excipient. In some embodiments, the formulation consists of about 3.0% or more of allantoin and a pharmaceutically acceptable excipient.

Embodiments describe a composition comprising allantoin in an amount from about 0.5% to about 15% by weight and a pharmaceutically acceptable excipient. In some embodiments, the composition comprises allantoin in an amount from about 1.5% to about 15% by weight and a pharmaceutically acceptable excipient. In some embodiments, the composition comprises allantoin in an amount from about 2.0% to about 15% by weight and a pharmaceutically acceptable excipient. In some embodiments, the composition comprises allantoin in an amount from about 2.5% to about 15% by weight and a pharmaceutically acceptable excipient. In some embodiments, the composition comprises allantoin in an amount from about 3.0% to about 15% by weight and a pharmaceutically acceptable excipient. In some embodiments, the composition comprises allantoin in an amount from about 3.0% to about 10% by weight and a pharmaceutically acceptable excipient. In some embodiments, the composition comprises allantoin in an amount from about 3.0% to about 9.0% by weight and a pharmaceutically acceptable excipient. In some embodiments, the composition comprises allantoin in an amount from about 3.0% to about 6.0% by weight and a pharmaceutically acceptable excipient. In some embodiments, the composition comprises allantoin in an amount from about 6.0% to about 15.0% by weight and a pharmaceutically acceptable excipient. In some embodiments, the composition comprises allantoin in an amount from about 6.0% to about 10.0% by weight and a pharmaceutically acceptable excipient. In some embodiments, the composition comprises allantoin in an amount from about 6.0% to about 9.0% by weight and a pharmaceutically acceptable excipient.

Embodiments describe a composition consisting essentially of allantoin in an amount from about 0.5% to about 15% by weight and a pharmaceutically acceptable excipient. In some embodiments, the composition consists essentially of allantoin in an amount from about 1.5% to about 15% by weight and a pharmaceutically acceptable excipient. In some embodiments, the composition consists essentially of allantoin in an amount from about 2.0% to about 15% by weight and a pharmaceutically acceptable excipient. In some embodiments, the composition consists essentially of allantoin in an amount from about 2.5% to about 15% by weight and a pharmaceutically acceptable excipient. In some embodiments, the composition consists essentially of allantoin in an amount from about 3.0% to about 15% by weight and a pharmaceutically acceptable excipient. In some embodiments, the composition consists essentially of allantoin in an amount from about 3.0% to about 10% by weight and a pharmaceutically acceptable excipient. In some embodiments, the composition consists essentially of allantoin in an amount from about 3.0% to about 9.0% by weight and a pharmaceutically acceptable excipient. In some embodiments, the composition consists essentially of allantoin in an amount from about 3.0% to about 6.0% by weight and a pharmaceutically acceptable excipient. In some embodiments, the composition consists essentially of allantoin in an amount from about 6.0% to about 15.0% by weight and a pharmaceutically acceptable excipient. In some embodiments, the composition consists essentially of allantoin in an amount from about 6.0% to about 10.0% by weight and a pharmaceutically acceptable excipient. In some embodiments, the composition consists essentially of allantoin in an amount from about 6.0% to about 9.0% by weight and a pharmaceutically acceptable excipient.

Embodiments describe a composition consisting of allantoin in an amount from about 0.5% to about 15% by weight and a pharmaceutically acceptable excipient. In some embodiments, the composition consists of allantoin in an amount from about 1.5% to about 15% by weight and a pharmaceutically acceptable excipient. In some embodiments, the composition consists of allantoin in an amount from about 2.0% to about 15% by weight and a pharmaceutically acceptable excipient. In some embodiments, the composition consists of allantoin in an amount from about 2.5% to about 15% by weight and a pharmaceutically acceptable excipient. In some embodiments, the composition consists of allantoin in an amount from about 3.0% to about 15% by weight and a pharmaceutically acceptable excipient. In some embodiments, the composition consists of allantoin in an amount from about 3.0% to about 10% by weight and a pharmaceutically acceptable excipient. In some embodiments, the composition consists of allantoin in an amount from about 3.0% to about 9.0% by weight and a pharmaceutically acceptable excipient. In some embodiments, the composition consists of allantoin in an amount from about 3.0% to about 6.0% by weight and a pharmaceutically acceptable excipient. In some embodiments, the composition consists of allantoin in an amount from about 6.0% to about 15.0% by weight and a pharmaceutically acceptable excipient. In some embodiments, the composition consists of allantoin in an amount from about 6.0% to about 10.0% by weight and a pharmaceutically acceptable excipient. In some embodiments, the composition consists of allantoin in an amount from about 6.0% to about 9.0% by weight and a pharmaceutically acceptable excipient.

In other embodiments, the formulation comprises more than about 1.5% by weight of allantoin, but not 1.5% or less of allantoin, and a pharmaceutically acceptable excipient. In some embodiments, the formulation comprises about 2.0% by weight or more of allantoin, but not 1.5% or less of allantoin, and a pharmaceutically acceptable excipient. In some embodiments, the formulation comprises about 2.5% by weight or more of allantoin, but not 1.5% or less of allantoin, and a pharmaceutically acceptable excipient. In some embodiments, the formulation comprises about 2.5% by weight or more of allantoin, but not less than 2.0% of allantoin, and a pharmaceutically acceptable excipient. In some embodiments, the formulation comprises about 3.0% by weight or more of allantoin, but not less than 2.5% of allantoin, and a pharmaceutically acceptable excipient. In some embodiments, the formulation comprises about 3.0% by weight or more of allantoin, but not less than 2.0% of allantoin, and a pharmaceutically acceptable excipient. In other embodiments, the formulation comprises about 3.0% by weight or more of allantoin, but not 1.5% or less of allantoin and a pharmaceutically acceptable excipient. In other embodiments, the formulation consists essentially of more than about 1.5% by weight of allantoin, but not 1.5% or less of allantoin, and a pharmaceutically acceptable excipient. In some embodiments, the formulation consists essentially of about 2.0% by weight or more of allantoin, but not 1.5% or less of allantoin, and a pharmaceutically acceptable excipient. In some embodiments, the formulation consists essentially of about 2.5% by weight or more of allantoin, but not 1.5% or less of allantoin, and a pharmaceutically acceptable excipient. In some embodiments, the formulation consists essentially of about 2.5% by weight or more of allantoin, but not less than 2.0% of allantoin, and a pharmaceutically acceptable excipient. In other embodiments, the formulation consists essentially of about 3.0% by weight or more of allantoin, but not less than 2.5% of allantoin, and a pharmaceutically acceptable excipient. In some embodiments, the formulation consists essentially of about 3.0% by weight or more of allantoin, but not less than 2.0% of allantoin and a pharmaceutically acceptable excipient. In other embodiments, the formulation consists essentially of about 3.0% by weight or more of allantoin but not 1.5% or less of allantoin and a pharmaceutically acceptable excipient. In other embodiments, the formulation consists of more than about 1.5% by weight of allantoin, but not 1.5% or less of allantoin, and a pharmaceutically acceptable excipient. In some embodiments, the formulation consists of about 2.0% by weight or more of allantoin, but not 1.5% or less of allantoin, and a pharmaceutically acceptable excipient. In some embodiments, the formulation consists of about 2.5% by weight or more of allantoin, but not 1.5% or less of allantoin, and a pharmaceutically acceptable excipient. In some embodiments, the formulation consists of about 2.5% by weight or more of allantoin, but not less than 2.0% of allantoin, and a pharmaceutically acceptable excipient. In some embodiments, the formulation consists of about 3.0% by weight or more of allantoin but not less than 2.5% of allantoin, and a pharmaceutically acceptable excipient. In some embodiments, the formulation consists of about 3.0% by weight or more of allantoin, but not less than 2.0% of allantoin, and a pharmaceutically acceptable excipient. In other embodiments, the formulation consists of about 3.0% by weight or more of allantoin, but not 1.5% or less of allantoin, and a pharmaceutically acceptable excipient.

Embodiments herein describe formulations of allantoin comprising an oil-in-water emulsion comprising allantoin, an emollient, an emulsifier and a solvent. In some embodiments, the formulation further comprises a pH modifier, a solubilizing agent, an antioxidant, a preservative, a chelating agent, an additive, a viscosity agent or a combination thereof. In some embodiments, the formulation comprises allantoin, an emollient, an emulsifier, a pH modifier, a solubilizing agent, an antioxidant, a preservative and a solvent. In some embodiments, the formulation consists essentially of allantoin, an emollient, an emulsifier, a pH modifier, a solubilizing agent, an antioxidant, a preservative and a solvent. In some embodiments, the formulation consists of allantoin, an emollient, an emulsifier, a pH modifier, a solubilizing agent, an antioxidant, a preservative and a solvent.

The formulations of various embodiments may include any number of additional components such as, for example, preservatives, emulsion stabilizers, pH adjusters, chelating agents, viscosity modifiers, antioxidants, surfactants, emollients, opacifying agents, skin conditioners, buffers, fragrances, and combinations thereof. In some embodiments, such additional components may provide a dual purpose. For example, certain surfactants may also act as emulsifiers, certain emollients may also act as viscosity modifiers, and certain buffering agents may also act as chelating agents.

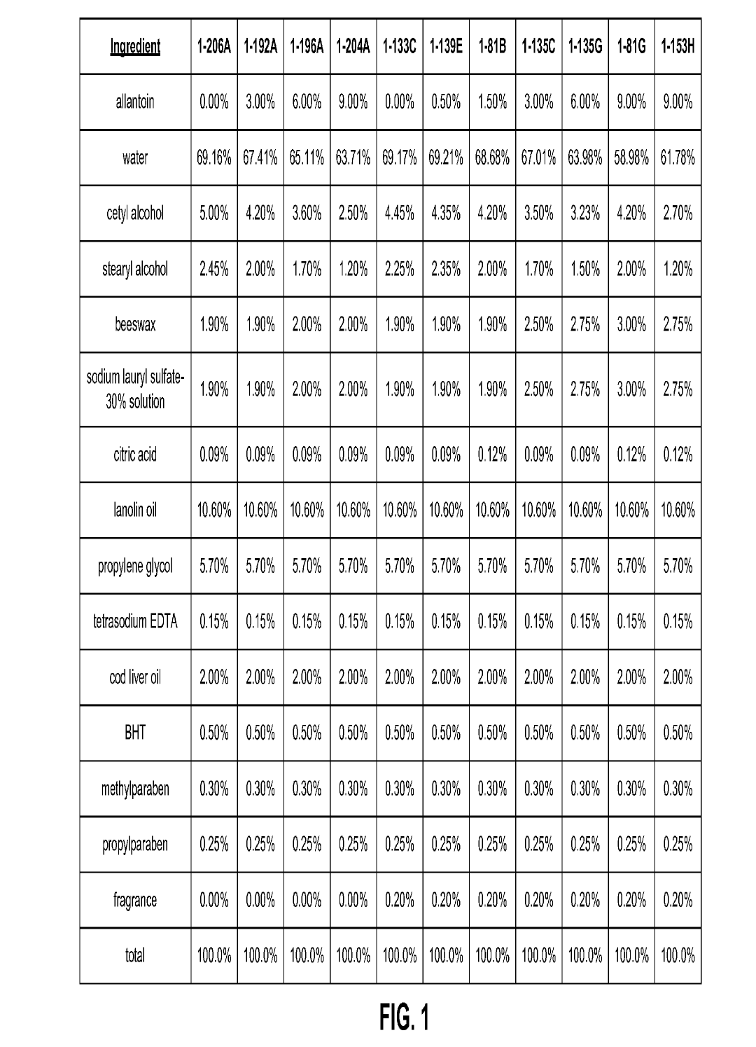

In particular, embodiments of the present disclosure relate to formulations of allantoin comprising an oil-in-water emulsion comprising allantoin; a solvent; an emollient such as, without limitation, lanolin oil, cod liver oil, mineral oil or an alcohol used as a thickening agent; an emulsifier such as, without limitation, sodium laurate sulfate or a white wax; an antioxidant such as, without limitation, butylated hydroxytoluene; a preservative such as, without limitation, methylparaben or propylparaben; a pH modifier such as, without limitation, citric acid or lactic acid; and a solubilizing agent such as, without limitation, glycerin or propylene glycol. In some embodiments, the formulation may further comprise a fragrance, an herbal extract, a viscosity agent such as, without limitation, cetyl alcohol or stearyl alcohol, a chelating agent such as, without limitation, tetrasodium EDTA, or a combination thereof. In some embodiments, the formulation of allantoin comprises any formulation disclosed in FIG. 1. In some embodiments, the formulation of allantoin consists essentially of any formulation disclosed in FIG. 1. In some embodiments, the formulation of allantoin consists of any formulation disclosed in FIG. 1. In some embodiments, the formulation of allantoin comprises a formulation selected from the group consisting of 1-192A, 1-196A, or 1-204A as shown in FIG. 1. In some embodiments, the formulation of allantoin consists essentially of a formulation selected from the group consisting of 1-192A, 1-196A, or 1-204A as shown in FIG. 1. In some embodiments, the formulation of allantoin consists of a formulation selected from the group consisting of 1-192A, 1-196A, or 1-204A as shown in FIG. 1. In an embodiment, a formulation of allantoin comprises an oil-in-water emulsion comprising allantoin, water, cetyl alcohol, stearyl alcohol, beeswax, sodium lauryl sulfate in a 30% solution, citric acid, lanolin oil, propylene glycol, tetrasodium EDTA, cod liver oil, butylated hydroxytoluene (BHT), methylparaben, and propylparaben.

In some embodiments, the formulation may include an emulsifying agent, or emulsifier. In embodiments, the emulsifier may be, for example, sodium lauryl sulfate, white waxes such as beeswax or paraffin wax, sesquioleates such as sorbitan sesquioleate or polyglyceryl-2-sesquioleate, ethoxylated esters of derivatives of natural oils such as the polyethoxylated ester of hydrogenated castor oil, silicone emulsifiers such as silicone polyols, anionic emulsifiers, fatty acid soaps such as potassium stearate and fatty acid sulphates like sodium cetostearyl sulphate, ethoxylated fatty alcohols, sorbitan esters, ethoxylated sorbitan esters, ethoxylated fatty acid esters such as ethoxylated stearates, ethoxylated mono, di-, and triglycerides, non-ionic self-emulsifying waxes, ethoxylated fatty acids, methylglucose esters such as polyglycerol-3 methyl glucose distearate, and combinations thereof. Various emulsions suitable for embodiments described herein and methods for preparing such emulsions are well known in the art and are described in, for example, Remington's Pharmaceutical Sciences, Mack Publishing Co., Easton, Pa., USA, which is hereby incorporated by reference in its entirety. In some embodiments, the formulation may include an emulsifier in an amount from about 1% to about 15%, and in other embodiments, the formulation may include from about 1% to about 10%, or from about 1% to about 5% emulsifier. If more than one emulsifier is used, the formulation may include from about 1% to about 5% or from about 1.5% to about 3% by weight of the formulation of each emulsifier.

In some embodiments, the formulations described herein may include one or more surfactants. Such embodiments are not limited by type of surfactant used; for example, in some embodiments, the one or more surfactants may be anionic surfactants such as alkyl sulfates, alkylether sulfates, alkylsulfonates, alkylaryl sulfonates, alkyl succinates, alkyl sulfosuccinates, N-alkoylsarcosinates, acyl taurates, acyl isethionates, alkyl phosphates, alkyl ether phosphates, alkyl ether carboxylates, α-olefinsulfonates, and the alkali metal and alkaline earth metal salts and ammonium and triethanolamine salts thereof. Such alkyl ether sulfates, alkyl ether phosphates and alkyl ether carboxylates can have between 1 and 10 ethylene oxide or propylene oxide units, and in some embodiments, 1 to 3 ethylene oxide units, per molecule. More specific examples include, but are not limited to, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzene sulfonate, triethanolamine dodecylbenzenesulfonate. In other embodiments, the one or more surfactants may be amphoteric surfactants such as, for example, alkylbetaines, alkylamidopropylbetaines, alkylsulfobetaines, alkylglycinates, alkylcarboxyglycinates, alkylamphoacetates or α-propionates, alkylamphodiacetates or α-dipropionates, and more specifically, cocodimethylsulfopropylbetaine, lauryl betaine, cocamidopropylbetaine or sodium cocamphopropionate.

In certain embodiments, the one or more surfactants may be non-ionic surfactants such as, for example, the reaction products of aliphatic alcohols or alkylphenols having 6 to 20 carbon atoms in a linear or branched alkyl chain with ethylene oxide and/or propylene oxide where the alkylene oxide may be from about 6 moles to about 60 moles per mole of alcohol. In particular embodiments, non-ionic surfactants may include alkylamine oxides, mono- and dialkylalkanolamides, fatty acid esters of polyethylenenglycols, ethoxylated fatty acids amides, saturated fatty acid alcohols reacted with ethylene oxide, alkyl polyglycosides, and sorbitan ether esters, and in some embodiments, the non-ionic surfactant may be ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-I4, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, and the like or combinations thereof, or one or more ceteareth in combination with a fatty acid alcohol such as stearyl alcohol, oleyl alcohol, linoleyl alcohol, arachidyl alcohol, cetyl alcohol, and the like. The surfactant of various embodiments may make up from about 0.1% to about 20% by weight of the formulation and in some embodiments, from about 0.5% to about 20% by weight of the formulation. In embodiments in which more than one surfactant is provided in the formulation, each surfactant may be from about 0.5% to about 10% by weight of the formulation, and in some embodiments, each surfactant of the formulation may be from about 0.5% to about 6% by weight of the formulation.

In some embodiments, the formulation may comprise emollients in an amount from about 8% to about 30% by weight of the formulation. In formulations that include more than one emollient, each emollient may be provided at about 0.05% to about 15% by weight of any one emollient. Emollients are well known in the art and are listed in, for example, the International Cosmetic Ingredient Dictionary, Eighth Edition, 2000, which is hereby incorporated by reference in its entirety. In certain embodiments, the emollient may be fatty esters, fatty alcohols, or combinations thereof including, but not limited to, diisopropyl adipate, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C₁₂ to C₁₆ fatty alcohol, C₁₂ to C₁₆ fatty alcohol lactate, isopropyl lanolate, 2-ethyl-hexyl salicylate, and combinations thereof. In some embodiments, the one or more emollients may be a combination of fatty alcohols. In certain embodiments, the one or more emollients may be 1-hexadecanol, acetylated lanolin, behenocyl dimethicone, C12-15 alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryl lactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and propylene glycol myristyl ether acetate. In some embodiments, the emollient may be a high molecular weight saturated and unsaturated fatty alcohol such as, but not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, or the like. In particular embodiments, the emollient may be selected from cetyl alcohol, stearyl alcohol, lanolin oil, cod liver oil, mineral oil, or a combination thereof. In some embodiments, the formulation may comprise an emollient such as, without limitations, cetyl alcohol in an amount from about 2% to about 6%, stearyl alcohol in an amount from about 1% to about 3%, lanolin in an amount from about 5% to about 15%, cod liver oil (and/or mineral oil) in an amount from about 0.05% to about 5% or combinations thereof.

In some embodiments, the formulation may include one or more viscosity modifiers. In some embodiments, the formulation may comprise from about 1% to about 10% or from about 1% to about 6% of each viscosity modifier. The viscosity modifier of such embodiments may generally include a high molecular weight compound such as, for example, carboxyvinyl polymer, carboxymethyl cellulose, polyvinyl pyrrolidone, hydroxyethyl cellulose, methyl cellulose, natural gum such as gelatin and tragacanth gum, and various alcohols such as polyvinyl alcohol. In other embodiments, the viscosity modifier may include ethanol or isopropyl alcohol. In some embodiments, the viscosity modifier may be a high molecular weight saturated and unsaturated fatty alcohol such as, but not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, and the like, and in certain embodiments, the viscosity modifier may be cetyl alcohol, stearyl alcohol or a combination thereof. In some embodiments, the formulation may comprise a viscosity modifier such as, without limitations, cetyl alcohol in an amount from about 2% to about 6%, stearyl alcohol in an amount from about 1% to about 3%, or combinations thereof.

Formulations of embodiments herein may further include a preservative. For example, preservatives useful in embodiments may include, but are not limited to, pentylene glycol, ethylene diamine tetra acetate (EDTA) and its salts, chlorhexidine and its diacetate, dihydrochloride, digluconate derivatives, 1,1,1-trichloro-2-methyl-2-propanol, parachlorometaxylenol, polyhexamethylenebiguanide hydrochloride, dehydroacetic acid, diazolidinyl urea, 2,4-dichlorobenzyl alcohol, 4,4-dimethyl-1,3-oxazolidine, formaldehyde, glutaraldehyde, dimethylidantoin, imidazolidinyl urea, 5-chloro-2-methyl-4-isothiazolin-3-one, ortho-phenylphenol, benzyl alcohol, benzoic acid and its salts, 4-hydroxybenzoic acid and its methyl-, ethyl-, propyl-, isopropyl-, butyl-, isobutyl-esters (parabens), methylparaben, propylparaben, isopropylparabens, isobutylparabens, butylparabens, ethylparaben, trichlosan, 2-phenoxyethanol, phenyl mercuric acetate, quaternium-15, methylsalicylate, salicylic acid and its salts, sorbic acid and its salts, iodopropanyl butylcarbamate, calcium sorbate, zinc pyrithione, 5-bromo-Snitro-1,3-dioxane, 2-bromo-2-nitropropane-1,3-diol, sulfites, bisulfites, and benzalkonium chloride, phenoxyethanol, 2-phenoxyethanol, chloroxylenol, diazolidinyl urea, and combinations thereof. In certain embodiments, the formulation may include a combination of methylparaben and propylparaben. Preservatives may be provided in any concentration known in the art. For example in some embodiments, the formulation may include preservatives in an amount from about 0.01% to about 3% by weight; and, in embodiments, the formulation may include from about 0.05% to about 1% or from about 0.05% to about 0.5% by weight of any one preservative.

The formulations of various embodiments may further include a chelating agent or combination of chelating agents. Examples of the chelating agents useful in various embodiments include, but are not limited to, alanine, sodium polyphosphate, sodium methaphosphate, citric acid, phosphoric acid, tartaric acid, ethylenediamine tetra acetic acid (Edetate, EDTA) and derivatives and salts thereof, dihydroxyethyl glycine, and combinations thereof. In particular embodiments, the chelating agent may be tetrasodium EDTA. The chelating agents may be provided in any effective amount. For example, in some embodiments, the formulation may include from about 0.01% to about 2% by weight chelating agent, and in other embodiments, the formulation may include from about 0.05% to about 0.5% or from about 0.05% to about 0.35% by weight chelating agent.

The formulations of certain embodiments may include one or more antioxidants. Numerous antioxidants are known in the art, and any such antioxidant may be used to prepare the formulations described herein. Examples of suitable antioxidants include, but are not limited to, amino acids such as glycine, histidine, tyrosine, trytophan and derivatives thereof, imidazoles such as urocanic acid and derivatives thereof, peptides, such as D,L-carnosine, D-carnosine, L-carnosine and derivatives thereof such as anserine, carotinoids, carotenes such as α-carotone, β-carotene, lycopene, and derivatives thereof, chlorogenic acid and derivatives thereof, lipoic acid and derivatives thereof such as dihydrlipoic acid, aurothioglycose, propylthiouracil and other thiols such as thioredoxin, glutathione, cysteine, cystine, cystamine and glycosyl, N-acetyl, methyl, ethyl, propyl, amyl, butyl, lauryl, palmitoyl, oleyl, α-linoleyl, cholesteryl and glyceryl esters and salts thereof, dilauryl thiodipropionate, distearyl thiodipropionate, thiodipropionic acid and derivatives thereof such as esters, ethers, peptides, lipids, nucleotides, nucleosides, and salts, sulfoximine compounds such as buthionine sulfoximines, homocysteine sulfoximine, buthionine sulfones, penta-, hexa-, hepta-thionine sulfoximine, unsaturated fatty acids and derivatives thereof such as α-linolenic acid, linoleic acid, oleic acid, folic acid and derivatives thereof, ubiquinone and ubiquinol and derivatives thereof, vitamin C and derivatives thereof such as ascorbyl palmitate, magnesium ascorbyl phosphate, ascorbyl acetate, tocopherals and derivatives such as vitamin E acetate, vitamin A and derivatives such as vitamin A palmitate, vitamin B and derivatives thereof, coniferyl benzoate of benzoin resin, rutinic acid and derivatives thereof, α-glycosylrutin, ferulic acid, furfurylidene glucitol, carnosine, butyl hydroxytoluene, trihydroxy-butyrophenone, uric acid and derivatives thereof, mannose and derivatives thereof, superoxide dismutase, zinc and derivatives thereof such as ZnO, ZnSO₄, selenium and derivatives thereof such as selenium methionine, stilbene and derivatives thereof such as stilbene oxide, trans-stilbene oxide and the like. In some embodiments, the antioxidants may include vitamin B, nordihydroguaiaretic acid, butylated hydroxyanisole (BHA), butylated hydroxytoluene (BHT), propyl gallate, erythorbate acid, sodium erythorbate, ascorbir palmitate, and ascorbyl stearate, butyl hydroxyanisole, and gallic esters, and in particular embodiments, the one or more antioxidants may include BHT. The antioxidant may be provided in any suitable amount. For example in some embodiments, one or more antioxidants may be from about 0.001% to about 3% by weight of the formulation, and in other embodiments, the one or more antioxidants may be from about 0.01% to about 1% by weight of the formulation or from about 0.05% to about 1% by weight of the formulation.

In some embodiments, the formulation may include a solubilizing agent. In embodiments, the solubilizers may be, for example, hydrochloric acid, sodium hydroxide, glycine, cyclodextrin, liquid paraffin, hydrogenated castor oil, ethanol, glycerin, propylene glycol, dilute hydrochloric acid, hydrogenated oils, purified water, physiological saline, water for injection, Macrogol 4000, Polysorbate 80, or a combination thereof. In particular embodiments, the solubilizing agent may be propylene glycol, glycerin or a combination thereof. In embodiments, the solubilizing agent comprises from about 1% to about 20%, from about 1% to about 10% or from about 2% to about 8% by weight of the formulation.

In certain embodiments, the formulation may include one or more opacifying agents. In some embodiments, components such as, for example, emollients, surfactants, and/or emulsifiers may provide sufficient opaqueness. In other embodiments, an additional opacifying agent may be provided to the formulation. Opacifying agents are well known in the art and include, but are not limited to, higher fatty alcohols such as cetyl, stearyl, cetostearyl alcohol, arachidyl and behenyl alcohols, solid esters such as cetyl palmitate, glyceryl laurate, stearamide MEA-stearate, high molecular weight fatty amides and alkanolamides and various fatty acid derivatives such as propylene glycol and polyethylene glycol esters. In other embodiments, opacifying agents may include inorganic materials such as, for example, magnesium aluminum silicate, zinc oxide, titanium dioxide and other sun-blocking agents. In embodiments in which an opacifying agent is used, the opacifying agent may be provided in any amount necessary to provide the desired opaqueness. In such embodiments, the opacifying agent may generally be from about 0.01% to about 20% by weight of the formulation, and in some embodiments, the opacifying agent may be from about 0.01% to about 10% or about 0.02% to about 5% by weight of the formulation.

In some embodiments, the formulation may include one or more skin conditioners. Common skin conditioners include, for example, mineral oil, petrolatum, aliphatic alcohols, lanolin and its derivatives, fatty acids, glycol fatty acids, sugars, glycerin, propylene glycol, sorbitols, and polyethylene glycols, vitamins and herbal derivatives. Additional skin conditioners can be found in CTFA Cosmetic Ingredient Handbook, 1st Ed., 1988, which is hereby incorporated herein by reference in its entirety. In some embodiments, the one or more skin conditioners may include, but are not limited to, humectants, such as fructose, glucose, glycerin, propylene glycol, glycereth-26, mannitol and urea, pyrrolidone carboxylic acid, hydrolyzed lecithin, coco-betaine, cysteine hydrochloride, glutamine, polyoxypropylene (15) polyoxyethylene (PPG-15), sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E and D, amino-functional silicones, ethoxylated glycerin, α-hydroxy acids and salts thereof, water-soluble fatty oil derivatives, such as PEG-24 hydrogenated lanolin, almond oil, grape seed oil and castor oil; numerous other water-soluble skin conditioners listed, and combinations thereof. In certain embodiments, the skin conditioners may include lanolin or lanolin derivatives, caprylic capric/triglyceride, diisopropyl adipate, and combinations thereof. Skin conditioners may be provided to various embodiments in any amount known in the art, and the amount of skin conditioner provided may vary depending upon the type of skin condition or combination of skin conditioners used. In general, the formulations of embodiments may include a conditioner in an amount from about 1% to about 30% by weight of the formulation or from about 1% to about 25% by weight of the formulation.

The pH of various embodiments may be of neutral to mildly acidic pH to allow for comfortable application to a subject's skin, particularly in light of the disease state or condition suffered by the subject. For example, in various embodiments, the pH of the formulations may be from about 2.5 to about 7.0, from about 4.0 to about 7.0, or from about 4.0 to about 5.5 at room temperature. In other embodiments, the pH of such formulations may be about 4.0 to about 5.0 at room temperature. Any components or combination of components known and useful in the art may be used to achieve an appropriate pH such as, for example, pH regulators including, but not limited to, lactic acid, citric acid, sodium citrate, glycolic acid, succinic acid, phosphoric acid, monosodium phosphate, disodium phosphate, oxalic acid, DL-malic acid, calcium carbonate, sodium hydroxide and sodium carbonate, sodium hydrogen carbonate, and ammonium hydrogen carbonate. In particular embodiments, the formulation may include, for example, citric acid or lactic acid as a pH modifier. In embodiments, the pH modifier may comprise from about 0.01% to about 1%, from about 0.05% to about 0.5%, from about 0.06% to about 0.15%, from about 0.06% to about 0.11%, or from about 0.06% to about 0.1% by weight of the formulation.

In embodiments, the formulation may further comprise a solvent. In some embodiments, the solvent may include one or more ingredients therein, with water being preferred in certain embodiments. Generally, the quantity of water used as a solvent may depend on the various other ingredients used. The solvent may be present in certain embodiments in a range of from about 10% to about 95% by weight, with certain embodiments including from about 40% to about 90%, from about 42% to about 87%, from about 42% to about 80%, from about 42% to about 75%, from about 42% to about 70%, or from about 42% to about 68% by weight of the formulation. The exact quantity of solvent may be dependent on the form of the product. For example, a product in lotion form may in certain preferred embodiments include more water than a product in spray form and a product in cream or butter form may include less water than a product in spray form. Deionized water is generally preferred. Other suitable solvent materials may also be used.

In embodiments, the formulation of embodiments herein may be physically and chemically stable. In some embodiments, the formulation of embodiments herein may be resistant to microbial agents for up to 4 years, up to 6 years, up to 8 years, up to 10 years, up to 12 years or up to 20 years. In some embodiments, the formulation of embodiments herein may be resistant to microbial agents for from about 4 to about 20 years, from about 4 to about 12 years, from about 4 to about 10 years, from about 4 to about 8 years, from about 4 to about 6 years, from about 6 to about 20 years, from about 6 to about 12 years, from about 6 to about 10 years, from about 6 to about 8 years, from about 8 to about 20 years, from about 8 to about 12 years, or from about 8 to about 10 years.

One embodiment relates to formulations of allantoin comprising an oil-in-water emulsion comprising about 3.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In further embodiments, the formulation consists essentially of about 3.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In certain embodiments, the formulation consists of about 3.0% of allantoin, water, cetyl alcohol, stearyl alcohol, beeswax, sodium lauryl sulfate in a 30% solution, citric acid, lanolin oil, propylene glycol, tetrasodium EDTA, cod liver oil, butylated hydroxytoluene, methylparaben, and propylparaben. In certain embodiments, the formulation further includes a fragrance. In some embodiments, the fragrance comprises from about 0.01% to about 5%, from about 0.01% to about 3%, from about 0.01% to about 2%, from about 0.01% to about 1% from about 0.01% to about 0.5%, from about 0.05% to about 3%, from about 0.05% to about 2%, from about 0.05% to about 1% from about 0.05% to about 0.5% by weight of the formulation. In certain embodiments, the formulation does not contain a fragrance. In embodiments, the formulation may further include an herbal extract. In certain embodiments, the formulation does not contain any herbal extracts.

In another embodiment, formulations of allantoin comprising an oil-in-water emulsion comprising about 6.0% of allantoin, water, cetyl alcohol, stearyl alcohol, beeswax, sodium lauryl sulfate in a 30% solution, citric acid, lanolin oil, propylene glycol, tetrasodium EDTA, cod liver oil, butylated hydroxytoluene, methylparaben, and propylparaben are provided. In certain embodiments, the formulation does not contain a fragrance. In certain embodiments, the formulation does not contain any herbal extracts. In further embodiments, the formulations consist essentially of about 6.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In certain embodiments, the formulations consist of about 6.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In certain embodiments, the formulation further includes a fragrance. In certain embodiments, the formulation does not contain a fragrance. In embodiments, the formulation may further include an herbal extract. In certain embodiments, the formulation does not contain any herbal extracts.

In another embodiment, formulations of allantoin comprising an oil-in-water emulsion comprising about 9.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben are provided. In certain embodiments, the formulation does not contain a fragrance. In certain embodiments, the formulation does not contain any herbal extracts. In further embodiments, the formulation consists essentially of about 9.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In certain embodiments, the formulation consists of about 9.0% of allantoin, water, cetyl alcohol, stearyl alcohol, beeswax, sodium lauryl sulfate in a 30% solution, citric acid, lanolin oil, propylene glycol, tetrasodium EDTA, cod liver oil (and/or mineral oil), butylated hydroxytoluene, methylparaben, and propylparaben. In certain embodiments, the formulation further includes a fragrance. In certain embodiments, the formulation does not contain a fragrance. In embodiments, the formulation may further include an herbal extract. In certain embodiments, the formulation does not contain any herbal extracts.

In another embodiment, the formulation comprises about 3.0% allantoin; about 67.01% water; about 3.5% cetyl alcohol; about 1.7% stearyl alcohol; about 2.5% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.5% sodium lauryl sulfate in a 30% solution. In further embodiments, the formulations consist essentially of about 3.0% allantoin; about 67.01% water; about 3.5% cetyl alcohol; about 1.7% stearyl alcohol; about 2.5% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.5% sodium lauryl sulfate in a 30% solution. In certain embodiments, the formulations consist of about 3.0% allantoin; about 67.01% water; about 3.5% cetyl alcohol; about 1.7% stearyl alcohol; about 2.5% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.5% sodium lauryl sulfate in a 30% solution.

In another embodiment, the formulation comprises about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.9% sodium lauryl sulfate in a 30% solution. In further embodiments, the formulation consists essentially of about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.9% sodium lauryl sulfate in a 30% solution. In certain embodiments, the formulation consists of about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.9% sodium lauryl sulfate in a 30% solution.

In another embodiment, the formulation comprises about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution. In further embodiments, the formulations consist essentially of about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution. In certain embodiments, the formulations consist of about 3.0% allantoin; about 67.41% water; about 4.2% cetyl alcohol; about 2% stearyl alcohol; about 1.9% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution.

In another embodiment, the formulation comprises about 3.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In further embodiments, the formulation consists essentially of about 3.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In certain embodiments, the formulation consists of about 3.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution.

In another embodiment, the formulation comprises about 6.0% allantoin; about 63.98% water; about 3.23% cetyl alcohol; about 1.5% stearyl alcohol; about 2.75% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution. In further embodiments, the formulations consist essentially of about 6.0% allantoin; about 63.98% water; about 3.23% cetyl alcohol; about 1.5% stearyl alcohol; about 2.75% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution. In certain embodiments, the formulations consist of about 6.0% allantoin; about 63.98% water; about 3.23% cetyl alcohol; about 1.5% stearyl alcohol; about 2.75% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution.

In another embodiment, the formulation comprises about 6.0% allantoin; about 64.81% water; about 3.5% cetyl alcohol; about 1.5% stearyl alcohol; about 2.3% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.3% sodium lauryl sulfate in a 30% solution. In further embodiments, the formulations consist essentially of about 6.0% allantoin; about 64.81% water; about 3.5% cetyl alcohol; about 1.5% stearyl alcohol; about 2.3% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.3% sodium lauryl sulfate in a 30% solution. In certain embodiments, the formulations consist of about 6.0% allantoin; about 64.81% water; about 3.5% cetyl alcohol; about 1.5% stearyl alcohol; about 2.3% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.3% sodium lauryl sulfate in a 30% solution.

In another embodiment, the formulation comprises about 6.0% allantoin; about 65.11% water; about 3.6% cetyl alcohol; about 1.7% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In further embodiments, the formulations consist essentially of about 6.0% allantoin; about 65.11% water; about 3.6% cetyl alcohol; about 1.7% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In certain embodiments, the formulations consist of about 6.0% allantoin; about 65.11% water; about 3.6% cetyl alcohol; about 1.7% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution.

In another embodiment, the formulation comprises about 6.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution. In further embodiments, the formulations consist essentially of about 6.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution. In certain embodiments, the formulations consist of about 6.0% allantoin; water; cetyl alcohol; stearyl alcohol; beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 1.5% sodium lauryl sulfate in a 30% solution.

In another embodiment, the formulation comprises about 9.0% allantoin; about 61.78% water; about 2.7% cetyl alcohol; about 1.2% stearyl alcohol; about 2.75% beeswax; about 0.12% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution. In further embodiments, the formulations consist essentially of about 9.0% allantoin; about 61.78% water; about 2.7% cetyl alcohol; about 1.2% stearyl alcohol; about 2.75% beeswax; about 0.12% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution. In certain embodiments, the formulations consist of about 9.0% allantoin; about 61.78% water; about 2.7% cetyl alcohol; about 1.2% stearyl alcohol; about 2.75% beeswax; about 0.12% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; about 0.2% fragrance; and about 2.75% sodium lauryl sulfate in a 30% solution.

In another embodiment, the formulation comprises about 9.0% allantoin; about 63.71% water; about 2.5% cetyl alcohol; about 1.2% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In further embodiments, the formulations consist essentially of about 9.0% allantoin; about 63.71% water; about 2.5% cetyl alcohol; about 1.2% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution. In certain embodiments, the formulations consist of about 9.0% allantoin; about 63.71% water; about 2.5% cetyl alcohol; about 1.2% stearyl alcohol; about 2.0% beeswax; about 0.09% citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and about 2.0% sodium lauryl sulfate in a 30% solution.

In another embodiment, the formulation comprises about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and 1.5% sodium lauryl sulfate in a 30% solution. In further embodiments, the formulations consist essentially of about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and 1.5% sodium lauryl sulfate in a 30% solution. In certain embodiments, the formulations consist of about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and 1.5% sodium lauryl sulfate in a 30% solution.

In another embodiment, the formulation comprises about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and sodium lauryl sulfate in a 30% solution. In further embodiments, the formulations consist essentially of about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and sodium lauryl sulfate in a 30% solution. In certain embodiments, the formulations consist of about 9.0% allantoin; water; cetyl alcohol; stearyl alcohol; beeswax; citric acid; about 10.6% lanolin oil; about 5.7% propylene glycol; about 0.15% tetrasodium EDTA; about 2% cod liver oil (and/or mineral oil); about 0.5% butylated hydroxytoluene; about 0.3% methylparaben; about 0.25% propylparaben; and sodium lauryl sulfate in a 30% solution.

Embodiments herein are also directed to methods of treating skin burns comprising administering a composition comprising an oil-in-water emulsion comprising allantoin in an amount from about 0.5% to about 15% by weight and a pharmaceutically acceptable excipient. In some embodiments, the formulation of allantoin comprises an oil-in-water emulsion comprising allantoin, an emollient, an emulsifier and a solvent. In some embodiments, the formulation further comprises a pH modifier, a solubilizing agent, an antioxidant, a preservative, a chelating agent, an additive, a viscosity agent or a combination thereof. In some embodiments, the formulation comprises allantoin, an emollient, an emulsifier, a pH modifier, a solubilizing agent, an antioxidant, a preservative and a solvent. In some embodiments, the formulation consists essentially of allantoin, an emollient, an emulsifier, a pH modifier, a solubilizing agent, an antioxidant, a preservative and a solvent. In some embodiments, the formulation consists of allantoin, an emollient, an emulsifier, a pH modifier, a solubilizing agent, an antioxidant, a preservative and a solvent. In some embodiments, a method of treating skin burns in a patient in need thereof comprises administering a formulation comprising allantoin, a solvent, an emollient, an emulsifier, an antioxidant, a preservative, a pH modifier and a solubilizing agent, wherein the allantoin is present in an amount of about 0.5% to about 15% by weight. In some embodiments, a method of treating skin burns in a patient in need thereof comprises administering a formulation consisting essentially of allantoin, a solvent, an emollient, an emulsifier, an antioxidant, a preservative, a pH modifier and a solubilizing agent, wherein the allantoin is present in an amount of about 0.5% to about 15% by weight. In some embodiments, a method of treating skin burns in a patient in need thereof comprises administering a formulation consisting of allantoin, a solvent, an emollient, an emulsifier, an antioxidant, a preservative, a pH modifier and a solubilizing agent, wherein the allantoin is present in an amount of about 0.5% to about 15% by weight.

In another embodiment, a method of treating skin burns comprises administering a formulation of allantoin comprising an oil-in-water emulsion comprising allantoin, an emollient, an emulsifier, a solvent and a pharmaceutically acceptable excipient. In some embodiments, the formulation further comprises a pH modifier, a solubilizing agent, an antioxidant, a preservative, a chelating agent, an additive, a viscosity agent or a combination thereof. In some embodiments, the formulation comprises allantoin, an emollient, an emulsifier, a pH modifier, a solubilizing agent, an antioxidant, a preservative, a solvent and a pharmaceutically acceptable excipient. In some embodiments, the formulation consists essentially of allantoin, an emollient, an emulsifier, a pH modifier, a solubilizing agent, an antioxidant, a preservative, a solvent and a pharmaceutically acceptable excipient. In some embodiments, the formulation consists of allantoin, an emollient, an emulsifier, a pH modifier, a solubilizing agent, an antioxidant, a preservative, a solvent and a pharmaceutically acceptable excipient. In some embodiments, a method of treating skin burns in a patient in need thereof comprises administering a formulation comprising allantoin, a solvent, an emollient, an emulsifier, an antioxidant, a preservative, a pH modifier, a solubilizing agent and a pharmaceutically acceptable excipient, wherein the allantoin is present in an amount of about 0.5% to about 15% by weight. In some embodiments, a method of treating skin burns in a patient in need thereof comprises administering a formulation consisting essentially of allantoin, a solvent, an emollient, an emulsifier, an antioxidant, a preservative, a pH modifier, a solubilizing agent and a pharmaceutically acceptable excipient, wherein the allantoin is present in an amount of about 0.5% to about 15% by weight. In some embodiments, a method of treating skin burns in a patient in need thereof comprises administering a formulation consisting of allantoin, a solvent, an emollient, an emulsifier, an antioxidant, a preservative, a pH modifier, a solubilizing agent and a pharmaceutically acceptable excipient, wherein the allantoin is present in an amount of about 0.5% to about 15% by weight. In certain embodiments, the pH of the formulation is about 3.0 to about 6.0, about 3.5 to about 6.0, about 4.0 to about 5.5, about 4.0 to about 5.0, or about 4.0 to about 4.5.

Embodiments of the present disclosure also relate to the use of formulations of allantoin in connection with excipients or stabilizers. Stabilizers include carbohydrates, amino acids, fatty acids, and surfactants and are known to those skilled in the art.

Compositions according to the embodiments described herein can contain other, optional ingredients. For example, compositions according to the present embodiments can contain glycerin, lactic acid, lipid-soluble components such as, but not limited to, caprylic/capric triglycerides; steareth-2; steareth-21; polyglyceryl-3 beeswax; a branched-carboxylic acid ester of a branched-chain alcohol selected from the group consisting of isononyl isononanoate, isodecyl isononanoate, isooctyl isononanotate, isooctyl isooctanoate, isononyl isooctanoate, isodecyl isooctanoate, isononyl isodecanoate, isooctyl isodecanoate, and isodecyl isodecanoate; an acrylates/C₁₀-C₃₀ alkyl acrylates cross-polymer; methylgluceth-20; a glyceryl ester of a long chain fatty acid selected from the group consisting of glyceryl monostearate, glyceryl monopalmitate, and glyceryl monoarachidate; hydrogenated vegetable oil; squalane; C₁₂-C₁₅ alkyl benzoates; di-C₁₂-C₁₅ alkyl fumarate; cholesterol; lanolin alcohol; octyldodecanol, isostearic acid; a branched-chain neopentanoate selected from the group consisting of octyldodecyl neopentanoate, heptyldodecyl neopentanoate, nonyldodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate; an arachidyl ester of a short-chain carboxylic acid selected from the group consisting of arachidyl propionate, arachidyl acetate, arachidyl butyrate, and arachidyl isobutyrate; a long-chain fatty acid ester of a medium-chain alcohol selected from the group consisting of octyl palmitate, octyl myristate, octyl stearate, heptyl palmitate, heptylmyristate, heptyl stearate, nonyl palmitate, nonyl myristate, and nonyl stearate; jojoba oil; a myristyl ester of a long-chain fatty acid selected from the group consisting of myristyl myristate, myristyl laurate, and myristyl palmitate; bisabolol; hydrogenated jojoba oil; jojoba esters; methyl-gluceth-20 sesquistearate; PPG-14 butyl ether; PPG-15 stearyl ether; PPG-1-isoceteth-3-accetate; laureth-2-benzoate; diisostearyl dimmer dilinoleate; a long-chain cis-monounsaturated fatty acid ester of a medium-chain alcohol; a medium-chain saturated carboxylic acid ester of a long-chain alcohol; hydrogenated soy glycerides; a long-chain fatty acid ester of cetyl alcohol selected from the group consisting of cetyl palmitate, cetyl stearate, and cetyl myristate; palm kernel oil; palm oil; and an arachidyl ester such as arachidyl acetate, arachidyl propionate, arachidyl butyrate, or arachidyl isobutyrate.

In addition, the composition can further comprise other ingredients that are generally used in the cosmetic art and in the art of over-the-counter skin preparations. These ingredients include, but are not limited to: (1) other plant extracts, such as horsetail extract, horse chestnut extract, rose extract, or lavender extract; (2) a short-chain carboxylic acid ester of tocopherol selected from the group consisting of tocopheryl acetate, tocopheryl propionate, tocopheryl butyrate, and tocopheryl isobutyrate; (3) a long-chain fatty acid ester of ascorbic acid selected from the group consisting of ascorbyl myristate, ascorbyl palmitate, and ascorbyl stearate; (4) a long-chain fatty acid ester of retinol or a retinol derivative or analogue wherein the acyl moiety of the ester is selected from the group consisting of myristic acid, palmitic acid, and stearic acid; and (5) a sunscreen, which can be at least one compound selected from the group consisting of octyl methoxycinnamate, p-aminobenzoate, glyceryl p-aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, α-phenyl cinnamonitrile, butyl cinnamoylpyruvate, umbelliferone, methylacetoumbelliferone, esculetin, methylesculetin, daphnetin, esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxy-biphenyldisulfonates, p-hydroxybiphenyldisulfonates, 7-hydroxycoumarin, 7-methylcoumarin, 3-phenyl-coumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxyl-substituted benzophenones, methoxy-substituted benzophenones, uric acid, vilouric acid, tannic acid, tannic acid hexaethylether, hydroquinone, oxybenzone, sulisobenzone, dioxybenzone, benzoresorcinol, 2,2′,4,4′-tetrahydroxyb enzophenone, 2,2′-dihydroxy-4,4-dimethoxybenzophenone, octabenzone, butylmethoxydibenzoylmethane, etocrylene, and 4-isopropyldibenzoylmethane. Other ingredients can also optionally be included, such as colorants, pigments, opacifiers, and the like.

Methods according to the embodiments described herein provide rapid improvement, are well tolerated by patients, are easy to apply, and can be used alone or with other methods for treatment of skin burns.

In any of the foregoing embodiments, the composition can further include fragrance. The use of fragrance is well known in the art of over-the-counter drug formulation, and many suitable fragrances are known in the art. The stability and function of the composition is not altered by the presence or absence of fragrance. In many alternatives, it may be desirable to avoid the use of fragrance which may trigger allergic reaction in patients predisposed to such reactions. Accordingly, in certain embodiments, the composition excludes a fragrance.

The compositions can further include other ingredients, such as proteins, humectants, other preservatives, essential oils, other vitamins, colorants, hydroxyacids, other plant extracts, sunscreens, sodium hyaluronate, lipids, fatty acids, thickeners, panthenol, and the like. The use of such components is conventional in the over-the-counter drug art. Typical sunscreens are octyl methoxycinnamate and benzophenone-3.

In certain embodiments, the compositions of the application do not comprise cetearyl octanoate, cetearyl ethyl hexanoate, or hexanoic acid. In certain embodiments, the compositions of the application do not comprise copper or zinc salts and/or copper-zinc compounds or complexes.

Pharmaceutical compositions provided by the present disclosure may comprise formulations of allantoin and in certain embodiments, in purified form, together with a suitable amount of one or more pharmaceutically acceptable vehicles, so as to provide a composition for proper administration to a patient with a skin burn. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The present compositions may also contain wetting agents, emulsifying agents, and/or pH buffering agents. In addition, auxiliary, stabilizing, thickening, lubricating, and/or coloring agents may be used. Other examples of suitable pharmaceutical vehicles are described in the art (see, for example, “Remington's Pharmaceutical Sciences,” Lippincott Williams & Wilkins, 21st Edition, 2005).

Pharmaceutical compositions disclosed herein may be prepared by standard mixing techniques, such as are conventional in the cosmetic art and in the art of over-the-counter drug formulation for blending lipid-soluble components and water-soluble components. These mixing techniques include both manual and mechanical mixing, and include homogenization mixing and sweep mixing. The mixing techniques to be used can be chosen by one of ordinary skill in the art based on variables such as the viscosity of the components to be mixed and the volume of those components, as well as the relative proportion of lipid-soluble and water-soluble ingredients. The composition can be mixed in two or more batches, such as one batch containing lipid-soluble ingredients and another batch containing water-soluble ingredients, and the batches can then be mixed at the final state of preparation.

For example, pharmaceutical compositions disclosed herein may be manufactured by following these steps: (1) mix and heat water, 30% solution of sodium lauryl sulfate, propylene glycol, tetrasodium EDTA and citric acid in one container (“Container 1”); (2) in another container (“Container 2”), mix and heat lanolin oil, beeswax, stearyl alcohol and cetyl alcohol; (3) when both containers reach about 170-180° F., add contents of Container 2 to Container 1; (4) add cod liver oil and butyl hydroxytoluene (BHT); (5) mix for about thirty minutes; (6) add allantoin; (7) mix for about thirty minutes; (8) cool contents to about 120° F.; (9) add methylparaben and propylparaben; (10) mix for about ten minutes; (11) remove the mixer and insert the homogenizer; (12) activate the homogenizer for about five minutes; (13) remove the homogenizer and insert mixer; (14) mix for about thirty minutes while maintaining temperature range of about 115-120° F.; (15) continue mixing while contents are cooled to about 115° F.; (16) stop mixing when contents reach about 115° F.; (17) remove mixer and cover drum; (18) store cream overnight at room temperature; and (19) package the cream into finished product containers.

Pharmaceutical compositions may be formulated in a conventional manner using one or more physiologically acceptable carriers, diluents, excipients, or auxiliaries, which facilitate processing of allantoin and one or more pharmaceutically acceptable vehicles into formulations that can be used pharmaceutically. Proper formulation is dependent upon the route of administration chosen.

Pharmaceutical compositions provided by the present disclosure may be administered for therapeutic or prophylactic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, pharmaceutical compositions or the present disclosure may be administered to a patient susceptible to or otherwise at risk of a particular disease or infection. Hence, a prophylactically effective amount is an amount sufficient to prevent, hinder or retard a disease state or its symptoms.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the above-described compound and a suitable carrier can be topical dosage forms which include, but are not limited to, solutions, powders, fluid emulsions, fluid suspensions, semi-solids, ointments, pastes, creams, gels and jellies, and foams comprising an effective amount of a polymer or copolymer of the present embodiment. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The embodiments illustrating the methods and materials used may be further understood by reference to the following non-limiting examples.

### Example 1

### Study Testing Topical Allantoin in a Porcine Burn Model

The purpose of this study is to determine the effects of application of topical allantoin cream at 3%, 6% and 9% on skin burns using a known animal model.

Minipigs (3 males/group, one group for partial-thickness burns, one group for full-thickness burns), will be fasted overnight prior to application of burns. Animals will be anesthetized via injection of ketamine/zylazine/zolazepan and kept sedated via administration of isofluorane. Ringers lactate will be administered intravenously during the burn procedure. Skin on the sides of the animals will be shaved and depilated with shaving razors; all skin areas to be treated will be separated by 4 cm and located 4 cm away from the center of the back. Body temperature and oxygenation will be monitored during the procedure and post-operatively. Cephalosporin will be administered intravenously (iv) prior to surgery and levofloxacin tablets will be administered for 7 days post-operatively along with fentanyl patches for analgesia.

Contact burns will be administered by application for 30 seconds/site of aluminum bars heated to either 100° C. for partial-thickness burns or 200° C. for full-thickness burns at a pressure of 0.4 kg/cm². The size of the wounds will be standardized to 3×3 cm. After application of the heated bar, the wound will be dressed with a multi-layer mask dressing (to minimize rubbing of the minipigs against the pen enclosures) with or without SD-101 cream test formulations. The test formulations with 0%, 3%, 6% or 9% allantoin are detailed in FIG. 1 (1-206A, 1-192-A, 1-196A, and 1-204A, respectively). Wound dressings will be changed daily under anesthesia.

Each animal will receive burns consisting of no more than 15% of total body surface area, and the burn sites will be divided in two sites with no SD-101 cream and an equal number of sites that receive either 0%, 3%, 6% or 9% allantoin cream in sufficient quantity to cover the burn site prior to bandaging.

Daily observations of the animals will be taken. Clinical wound healing assessment will include wound healing as judged by re-epithelialization, hematomas and fibrin deposition, which will be assessed using a standardized scale (1-5). Assessment will be done when wound dressings are changed daily.

Standardized photographs will be taken 24 hours, 48 hours, 7 days and 14 days after creation of wounds and assessed via planimetry for total wound size.

Histopathology of the wound sites (dermal and epidermal), including assessment of the wound edges and wound center 14 days post-burn application following necropsy of the animals, will be performed. Standard H&E staining will be used.

Blood will be collected at 24 hours, 48 hours, 7 days and 14 days post-operation for standard clinical chemistry and hematology measurements.

Although the present invention has been described in considerable detail with reference to certain preferred embodiments thereof, other versions are possible. Accordingly, the present embodiments are to be considered as illustrative and not restrictive.

1. A method of treating a skin burn on a patient comprising administering to the burn a composition comprising allantoin in an amount from about 2.5% to about 15% by weight and a pharmaceutically acceptable excipient.

2. The method of claim 1, wherein the skin burn is caused by heat, electricity, chemicals, friction, radiation, or combinations thereof.

3. The method of claim 2, wherein the skin burn is caused by heat.

4. The method of any one of claims 1-3, wherein the skin burn is a first degree burn or a second degree burn.

5. The method of any one of claims 1-3, wherein the skin burn is a third degree burn or a third degree burn treated with a skin graft.

6. The method of any one of claims 1-5, wherein the composition is an oil-in-water emulsion further comprising an emollient and an emulsifier.

7. The method of claim 6, wherein the emollient is selected from the group consisting of lanolin oil, cod liver oil, mineral oil, an alcohol, and any combination thereof.

8. The method of claim 6, wherein the emulsifier is selected from the group consisting of sodium laurate sulfate, a white wax, and a combination thereof.

9. The method of any one of claims 1-8, wherein the composition further comprises a pH modifier, a solubilizing agent, an antioxidant, a preservative, a chelating agent, a viscosity agent or any combination thereof.

10. The method of claim 9, wherein the pH modifier is citric acid; the solubilizing agent is propylene glycol; the antioxidant is butylated hydroxytoluene (BHT); the preservative is selected from the group consisting of methylparaben, propylparaben, and a combination thereof; the chelating agent is tetrasodium EDTA; the viscosity enhancing agent is selected from the group consisting of cetyl alcohol, stearyl alcohol, and a combination thereof; and the pharmaceutically acceptable excipient is water.

11. The method of any one of claims 1-5, wherein the composition further comprises an emollient, an emulsifier, a solvent, a pH modifier, a solubilizing agent, an antioxidant, a preservative, a chelating agent, an additive, a viscosity agent, or any combination thereof.

12. The method of any one of claims 1-11, wherein the composition further comprises a fragrance.

13. The method of any one of claims 1-8, wherein the composition further comprises tetrasodium EDTA.

14. The method of claim 13, wherein the tetrasodium EDTA is in an amount from about 0.05% to about 0.5%.

15. The method of any one of claims 1-5, wherein the composition comprises: water, cetyl alcohol, stearyl alcohol, beeswax, sodium lauryl sulfate in an about 30% solution, citric acid, lanolin oil, propylene glycol, cod liver oil, butylated hydroxytoluene, methylparaben, and propylparaben.

16. The method of claim 15, wherein the composition comprises: water in an amount from about 40 to about 90%; cetyl alcohol in an amount from about 0.5 to about 15%; stearyl alcohol in an amount from about 1% to about 3%; beeswax in an amount from about 1.5% to about 3%; sodium lauryl sulfate in a 30% solution in an amount from about 1.5% to about 3%; citric acid in an amount from about 0.5% to about 0.2%; lanolin oil in an amount from about 5% to about 15%; propylene glycol in an amount from about 2% to about 8%; cod liver oil in an amount from about 0.05% to about 5%; butylated hydroxytoluene in an amount from about 0.05% to about 1%; methylparaben in an amount from about 0.05% to about 0.5%; and propylparaben in an amount from about 0.05% to about 0.5% by weight of the formulation.

17. The method of any one of claims 1-5, wherein the composition comprises: water; cetyl alcohol; stearyl alcohol; beeswax; sodium lauryl sulfate in an about 30% solution; citric acid; lanolin oil; glycerin; cod liver oil; butylated hydroxytoluene; methylparaben; and propylparaben.

18. The method of any one of claims 1-5, wherein the composition comprises: water; cetyl alcohol; stearyl alcohol; beeswax; sodium lauryl sulfate in an about 30% solution; lactic acid; lanolin oil; propylene glycol; cod liver oil; butylated hydroxytoluene; methylparaben; and propylparaben.

19. The method of any one of claims 1-5, wherein the composition comprises: water; cetyl alcohol; stearyl alcohol; beeswax; sodium lauryl sulfate in an about 30% solution; lactic acid; lanolin oil; glycerin; cod liver oil; butylated hydroxytoluene; methylparaben; and propylparaben.

20. The method of any one of claims 1-5, wherein the composition comprises: water; cetyl alcohol; stearyl alcohol; beeswax; sodium lauryl sulfate in an about 30% solution; citric acid; lanolin oil; propylene glycol; mineral oil; butylated hydroxytoluene; methylparaben; and propylparaben.

21. The method of any one of claims 1-20, wherein the composition has a pH in the range from about 4.0 to about 5.5.
